(19) United States
(12) Patent Application Publication
(10) Pub. No.: US 2024/0426828 A1
(43) Pub. Date: Dec. 26, 2024

(54) ROLE OF OXCT1 SUCCINYLTRANSFERASE ACTIVITY IN DIAGNOSIS AND TREATMENT OF TUMORS

(71) Applicant: INSTITUTE OF HEALTH AND MEDICINE, HEFEI COMPREHENSIVE NATIONAL SCIENCE CENTER, Hefei (CN)

(72) Inventors: Huafeng ZHANG, Hefei (CN); Ping GAO, Hefei (CN); Wenhao MA, Hefei (CN); Yuchen SUN, Hefei (CN)

(73) Assignee: INSTITUTE OF HEALTH AND MEDICINE, HEFEI COMPREHENSIVE NATIONAL SCIENCE CENTER, Hefei (CN)

(21) Appl. No.: 18/825,891

(22) Filed: Sep. 5, 2024

Related U.S. Application Data
(63) Continuation of application No. PCT/CN2023/127692, filed on Oct. 30, 2023.

(30) Foreign Application Priority Data
Dec. 27, 2022 (CN) 202211687445.2

(51) Int. Cl. G01N 33/574; G01N 33/573

(52) U.S. Cl. CPC G01N 33/57496; G01N 33/573; G01N 2333/91194

(57) ABSTRACT

Provided is the use of OXCT1 protein as succinyltransferase. It has been found that the OXCT1 protein serving as succinyltransferase can promote the growth of cancer cells by modulating succinylation modification of protein. Therefore, the design of related enzyme inhibitors can inhibit the activity of succinyltransferase, thereby inhibiting the occurrence and progression of cancer, thereby providing a new concept for targeted therapy.

### CROSS-REFERENCE TO RELATED APPLICATIONS

This application is a continuation of International Application No. PCT/CN2023/127692, filed on Oct. 30, 2023, which claims priority to Chinese Patent Application No. 202211687445.2, filed on Dec. 27, 2022. The disclosures of the aforementioned applications are hereby incorporated by reference in their entireties.

### FIELD

The present disclosure relates to the field of biomedicine. In particular, the present disclosure relates to the role of OXCT1 succinyltransferase activity in the diagnosis and treatment of tumors.

### BACKGROUND

Cancer, as one of the main causes of abnormal human death, is referred to as the first killer of mankind in the 21^{st} century. According to the global death data of 185 countries published by the World Health Organization in 2020, cancer is the first or second leading cause of death among people under the age of 70 in 112 countries. In the same year, the statistical data of the incidence and mortality of 36 cancer species in 185 countries from the International Agency for Research on Cancer of the World Health Organization indicate that up to 19.3 million new cases of cancer occurred globally in 2020. Among them, Asia increased by 9.51 million new cases of cancer, accounting for 49.3% of the global total, and the global number of cancer-caused deaths is close to 9.9 million, of which 5.77 million are in Asia, accounting for 58.3% of the total global deaths. Due to such an incidence and mortality of cancer, early diagnosis and prognosis are important to improve the survival rate of cancer patients.

Therefore, it is important in early diagnosis and timely treatment of cancer to develop new and efficient specific expression factors of cancer and elaborate its molecular mechanism in the progression of cancer.

### SUMMARY

In a first aspect of the present disclosure, the present disclosure provides a target for treating cancer. The target is the amino acid G424 of the OXCT1 protein.

In a second aspect of the present disclosure, a cancer therapeutic formulation is provided. The formulation includes a reagent inhibiting or blocking the above-mentioned target.

In a third aspect of the present disclosure, the present disclosure provides a method for determining a source of a sample to be tested. The method includes: determining the source of a sample to be tested based on a succinyltransferase activity of OXCT1 protein in the sample to be tested.

In a fourth aspect of the present disclosure, the present disclosure provides a system for determining the source of a sample to be tested. The system includes a detecting device configured to detect a succinyltransferase activity of OXCT1 protein in the sample to be tested; and a determining device connected to the detecting device and configured to determine the source of the sample to be tested based on the succinyltransferase activity of the OXCT1 protein obtained in the detecting device.

In a fifth aspect of the present disclosure, the present disclosure provides a method for treating cancer. The method includes: inhibiting or blocking the aforementioned target in cancer cells or inhibiting an expression or activity of the OXCT1 protein.

### BRIEF DESCRIPTION OF THE DRAWINGS

The foregoing and/or additional aspects and advantages of the present disclosure will become apparent and readily appreciated from the following description of the embodiments, in conjunction with the accompanying drawings.

FIG. 1 is a diagram of a system for determining a source of a sample to be tested according to an embodiment of the present disclosure;

FIG. 2A is a graph of a succinylation modification level of a protein subsequent to an overexpression of OXCT1 in different hepatoma cell lines detected by Western blot according to an embodiment of the present disclosure (EV represents a control group);

FIG. 2B is a graph of a succinylation modification level of total protein subsequent to OXCT1 knockdown in different hepatoma cell lines detected by Western blot according to an embodiment of the present disclosure (NTC represents a control group, and shOXCT1 represents an experimental group silencing OXCT1 with shRNA);

FIG. 3A is a graph of succinylation modification of a substrate LACTB by OXCT1 detected by co-immunoprecipitation combined with Western blot according to an embodiment of the present disclosure;

FIG. 3B is a graph of OXCT1 succinyltransferase activity in an in vitro succinyltransferase assay detected by Western blot according to an embodiment of the present disclosure;

FIG. 4 is a graph of the OXCT1 succinyltransferase active site identified by Western blot according to an embodiment of the present disclosure (where EV represents control and WT represents overexpression);

FIG. 5A is a graph of the tumorigenesis of mice for the effect of OXCT1 on the development of liver cancer detected by Western blot according to an embodiment of the present disclosure;

FIG. 5B is a statistical graph of a ratio of liver weight to body weight according to an embodiment of the present disclosure;

FIG. 5C is a statistical graph of the total number of foci of liver tumors according to an embodiment of the present disclosure;

FIG. 5D is a statistical graph of the number of foci of liver tumors with diameter >3 mm according to an embodiment of the present disclosure;

FIG. 5E is a statistical graph of the maximum tumor diameter of the liver according to an embodiment of the present disclosure;

FIG. 5F is a graph of the succinylation modification level of total protein of respective tumor tissues detected by Western blot according to an embodiment of the present disclosure;

FIG. 6A is a graph of a succinylation modification level of total protein detected by Western blot after an overexpression of OXCT1 variants or OXCT1 knockdown in colorectal cancer (EV and NTC represent control groups; WT, E344G, and G424E represent experimental groups overexpressing the respective variants; and shOXCT1 represents an experimental group silencing OXCT1 with shRNA);

FIG. 6B is a graph of a succinylation modification level of total protein detected by Western blot after an overexpression of OXCT1 variants or OXCT1 knockdown in breast cancer (EV and NTC represent control groups; WT, E344G, and G424E represent experimental groups overexpressing the respective variants; and shOXCT1 represents an experimental group silencing OXCT1 with shRNA);

FIG. 7A and FIG. 7B are graphs of a proliferation level of cells detected by cytometry after an overexpression of OXCT1 variants or OXCT1 knockdown in colorectal cancer (EV and NTC represent control groups; WT, E344G, and G424E represent experimental groups overexpressing the respective variants; and shOXCT1 represents an experimental group silencing OXCT1 with shRNA);

FIG. 8A and FIG. 8B are graph of a proliferation level of cells detected by cytometry after an overexpression of OXCT1 variants or OXCT1 knockdown in breast cancer (EV and NTC represent control groups; WT, E344G, and G424E represent experimental groups overexpressing the respective variants; and shOXCT1 represents an experimental group silencing OXCT1 with shRNA).

### DETAILED DESCRIPTION

The present disclosure aims to solve, at least to some extent, at least one of the technical problems existing in the prior art.

Ketone metabolism is the central node of physiological homeostasis. Ketones, including acetone, acetoacetic acid, and β-hydroxybutyric acid, are important alternative metabolic fuel sources for eukaryotes, prokaryotes, and archaea. Ketones also play an important role in maintaining cellular and human homeostasis. OXCT1 protein is a key enzyme in the breakdown of ketones, catalyzing the reversible transfer of coenzyme A (CoA) from succinyl-CoA to acetoacetate to form acetyl-CoA. The Applicant found the followings in the previous research: the OXCT1 protein is highly expressed in cancer cells under serum starvation conditions and promotes cancer cell proliferation through a ketone decomposing function; and the high expression of the OXCT1 protein is significantly associated with poor prognosis of patients. Recently, the Applicant found that the OXCT1 protein itself has succinyltransferase activity and that its succinyltransferase activity is independent of its classical ketolytic enzyme activity. That is, the OXCT1 protein, as a bifunctional enzyme, has both a ketone-metabolizing enzyme activity and a succinyltransferase activity.

The present disclosure provides the roles of the succinyltransferase activity of the OXCT1 protein in the occurrence and progression of cancer. Based on the succinyltransferase activity of the OXCT1 protein, the present disclosure provides a new concept in the analysis of molecular mechanisms of pathogenesis of cancer species with high expression of OXC1 such as liver cancer, pancreatic cancer, breast cancer, prostate cancer and colorectal cancer, and in the exploration of new targets and strategies for the diagnosis and treatment of these cancer species with high expression of OXCT1.

The Applicant found through cell biological experiments, molecular biological experiments and 4D proteomics experiments that the OXCT1 protein has the succinyltransferase activity. The Applicant found in tumor tissues and paracancerous tissues of cancer patients with high expression of OXCT1 protein and in mouse cancer models that the succinylation modification level of total protein in tumor tissues was significantly higher than that in paracancerous tissues. Further, the present disclosure has found that OXCT1 facilitates succinylation modification of protein substrates by its succinyltransferase function in in vitro systems. In addition, the present disclosure, in conjunction with the NRAS/shP53 mouse in situ cancer induction model, found that the succinyltransferase inactivation of the OXCT1 protein significantly inhibited the development of cancer. The above suggests that the succinyltransferase activity of the OXCT1 protein in the present disclosure has the potential to aid in the diagnosis, treatment, or prognostic evaluation of cancer.

In the present disclosure, it has been found that the OXCT1 protein has the succinyltransferase activity and that the OXCT1 protein promotes the occurrence and progression of related cancers depending on its succinyltransferase function. Specifically, it provides a new concept and method for clinical diagnosis and treatment of related cancers by inhibiting a level of the OXCT1 protein in cancer cells through a prepared targeted drug such as a neutralizing antibody, or by inhibiting the succinyltransferase activity of OXCT1 protein. Further, the detected and treated cancers according to the present disclosure are related to the cancer species with high expression of OXCT1 protein.

Thus, in an aspect of the present disclosure, the present disclosure provides use of OXCT1 protein as succinyltransferase. The Applicant has found that the OXCT1 protein serving as succinyltransferase can promote the growth of cancer cells by modulating a protein succinylation modification.

In another aspect of the present disclosure, the present disclosure provides use of an amino acid Glycine at position 424 (G424) of the OXCT1 protein as an enzyme active center of succinyltransferase. The Applicant has found that the succinyltransferase activity can be significantly inhibited by designing an enzyme inhibitor capable of adhering to and blocking a binding site of the enzyme active center, thereby further inhibiting the growth of cancer cells.

In yet another aspect of the present disclosure, the present disclosure provides a target for treating cancer. According to an embodiment of the present disclosure, the target is the amino acid G424 of the OXCT1 protein. The Applicant has found that the succinylation modification process regulated by amino acid G424 of the OXCT1 protein plays an important role in the proliferation and growth of cancer cells, providing a new concept for targeted therapy in the future.

According to an embodiment of the present disclosure, the target for treating cancer may further include at least one of the following additional technical features.

According to an embodiment of the present disclosure, the amino acid G424 is the succinyltransferase active site of the OXCT1 protein. The Applicant has found that related enzyme inhibitors can be designed to target at the enzyme active site to inhibit the succinyltransferase activity, further inhibiting the growth of cancer cells.

According to an embodiment of the present disclosure, the cancer is selected from cancer species with high expression of the OXCT1 protein. Therefore, the development of cancer can be inhibited by inhibiting the succinyltransferase activity of the OXCT1 protein.

According to an embodiment of the present disclosure, the cancer is at least one selected from liver cancer, colorectal cancer, and breast cancer. The Applicant has found that medicaments designed to target at the amino acid G424 of the OXCT1 protein have superior inhibitory effects on liver cancer, colorectal cancer, and breast cancer.

In a further aspect of the present disclosure, the present disclosure provides use of a reagent in the preparation of a cancer therapeutic formulation. According to an embodiment of the present disclosure, the reagent is used to inhibit or block the above-mentioned target. By using the reagent, the activity of the target can be effectively inhibited, thereby effectively inhibiting the occurrence and progression of cancer.

According to an embodiment of the present disclosure, the use of the reagent in the preparation of the cancer therapeutic formulation may further include at least one of the following additional technical features.

According to an embodiment of the present disclosure, the reagent includes at least one selected from a siRNA, a neutralizing antibody, and a compound inhibiting the expression of the OXCT1 protein.

According to an embodiment of the present disclosure, the cancer is selected from cancer species with high expression of OXCT1 protein. Therefore, it is possible to inhibit the development of cancer by inhibiting the succinyltransferase activity of the OXCT1 protein.

According to an embodiment of the present disclosure, the cancer is at least one selected from liver cancer, pancreatic cancer, colorectal cancer, and breast cancer.

According to an embodiment of the present disclosure, the cancer is at least one selected from liver cancer, colorectal cancer, and breast cancer. The Applicant has found that the therapeutic formulation designed to target at the amino acid G424 of the OXCT1 protein has superior inhibitory effects on liver cancer, colorectal cancer, and breast cancer.

In yet another aspect of the present disclosure, a cancer therapeutic formulation is provided. According to an embodiment of the present disclosure, the formulation includes a reagent inhibiting or blocking the above-mentioned target. With the formulation, the activity of the target can be effectively inhibited, and then the occurrence and progression of cancer can be effectively inhibited, providing a new concept for the subsequent targeted therapy.

According to an embodiment of the present disclosure, the cancer therapeutic formulation may further include at least one of the following additional technical features:

According to an embodiment of the present disclosure, the reagent includes at least one selected from a siRNA, a neutralizing antibody, and a compound inhibiting expression of the OXCT1 protein.

According to an embodiment of the present disclosure, the cancer is selected from cancer species with high expression of OXCT1 protein. Therefore, the development of cancer can be inhibited by inhibiting the succinyltransferase activity of the OXCT1 protein.

According to an embodiment of the present disclosure, the cancer is at least one selected from liver cancer, pancreatic cancer, colorectal cancer, and breast cancer.

According to an embodiment of the present disclosure, the cancer is at least one selected from liver cancer, colorectal cancer, and breast cancer.

In a further aspect of the present disclosure, the present disclosure provides use of a reagent in the preparation of a kit for inhibiting a succinyltransferase activity of OXCT1 protein. According to an embodiment of the present disclosure, the reagent is used to inhibit or block the above-mentioned target or to inhibit the expression or activity of the OXCT1 protein. The kit prepared by the reagent inhibiting or blocking the above-mentioned target according to the embodiments of the present disclosure can be used to inhibit the succinyltransferase activity of the OXCT1 protein.

In yet another aspect of the present disclosure, the present disclosure provides use of a reagent in the preparation of a formulation for inhibiting a succinyltransferase activity of OXCT1 protein. According to an embodiment of the present disclosure, the reagent is used to inhibit or block the above-mentioned target. The Applicant has found that the succinyltransferase activity of OXCT1 protein can be effectively inhibited by employing reagent inhibit or block the above-mentioned target.

In yet another aspect of the present disclosure, the present disclosure provides a method for determining a source of a sample to be tested. According to an embodiment of the present disclosure, the method includes: determining the source of a sample to be tested based on a succinyltransferase activity of OXCT1 protein in the sample to be tested.

According to an embodiment of the present disclosure, the method for determining the source of a sample to be tested may further include at least one of the following additional technical features.

According to an embodiment of the present disclosure, the succinyltransferase activity of the OXCT1 protein in the sample to be tested is determined based on a protein succinylation level in the sample to be tested. The detected protein succinylation level can effectively reflect the succinyltransferase activity in the sample to be tested.

According to an embodiment of the present disclosure, the protein succinylation level in the sample to be tested is at least two times a predetermined protein succinylation level and is an indication that the sample to be tested is derived from a cancer tissue. The Applicant has found that the amount of protein succinylation modification in cancer tissue is higher than that in normal tissue. Therefore, it can predict by detecting the protein succinylation level in the tissue whether the tissue is cancerous.

It should be noted that the “predetermined protein succinylation level” as used herein refers to the normal protein succinylation level in normal tissue, i.e., tissue that has not become cancerous.

According to an embodiment of the present disclosure, the protein succinylation level in the sample to be tested may be detected by at least one of Western blot, immunohistochemistry, immunofluorescence, and enzyme-linked immunosorbent assay.

According to an embodiment of the present disclosure, the cancer tissue is at least one selected from a liver cancer tissue, a pancreatic cancer tissue, a colorectal cancer tissue, and a breast cancer tissue.

According to an embodiment of the present disclosure, the cancer tissue is liver cancer, colorectal cancer, and breast cancer.

In a further aspect of the present disclosure, the present disclosure provides use of a reagent in the preparation of a kit detecting cancer. According to an embodiment of the present disclosure, the reagent is used to detect a succinyltransferase activity of OXCT1 protein in a sample to be tested. It can be quickly known by detecting the level of succinyltransferase activity of the OXCT1 protein in the sample to be tested with the reagent whether the sample to be tested is cancerous.

According to an embodiment of the present disclosure, the use of the reagent in the preparation of the kit may further include at least one of the following additional technical features.

According to an embodiment of the present disclosure, the reagent is a related reagent of Western blot, immunohistochemistry, immunofluorescence, or enzyme-linked immunosorbent assay.

Note that, the “related reagent” refers to a reagent that can be used to detect the succinyltransferase activity of the OXCT1 protein in a sample to be tested by the method such as Western blot, immunohistochemistry, immunofluorescence, or enzyme-linked immunosorbent assay.

In a further aspect, the present disclosure provides use of a device in the preparation of a system for detecting a cancer. The device is configured to detect a succinyltransferase activity of OXCT1 protein in a sample to be tested. It can be quickly known by detecting the level of succinyltransferase activity of the OXCT1 protein in the sample to be tested using the device whether the sample to be tested is cancerous.

According to an embodiment of the present disclosure, the device is configured to obtain a protein succinylation level in the sample to be tested by performing Western blot, immunohistochemistry, immunofluorescence, or enzyme-linked immunosorbent assay.

In yet another aspect of the present disclosure, the present disclosure provides a system for determining the source of a sample to be tested. According to an embodiment of the present disclosure, the system includes a detecting device configured to detect a succinyltransferase activity of OXCT1 protein in the sample to be tested; and a determining device connected to the detecting device and configured to determine the source of the sample to be tested based on the succinyltransferase activity of the OXCT1 protein obtained in the detecting device. The system according to the embodiments of the present disclosure can implement the above-mentioned method for determining the source of a sample to be tested, and thus it can effectively detect the succinyltransferase activity of the OXCT1 protein in the sample to be tested, thereby providing the foundation for subsequent scientific workers to continue to analyze the sample to be tested. Alternatively, the system can diagnose or evaluate whether cancer occurs clinically, assisting doctors to accurately predict the progression of diseases and timely perform clinical intervention.

According to an embodiment of the present disclosure, the system for determining the source of a sample to be tested may further include at least one of the following additional technical features.

According to an embodiment of the present disclosure, the detecting device is configured to determine, through obtaining a protein succinylation level in the sample to be tested, the succinyltransferase activity of the OXCT1 protein. The succinyltransferase activity in the sample to be tested can be effectively reflected by detecting the protein succinylation level.

According to an embodiment of the present disclosure, the detecting device is configured to perform at least one of Western blot, immunohistochemistry, immunofluorescence, and enzyme-linked immunosorbent assay.

According to an embodiment of the present disclosure, the protein succinylation level in the sample to be tested is at least two times a predetermined protein succinylation level and is an indication that the sample to be tested is derived from a cancer tissue. The Applicant has found that the amount of succinylated protein modification in cancer tissue is higher than that in normal tissue, and thus it is possible to predict whether the tissue is cancerous by detecting the succinylation modification level of protein in the tissue.

In yet another aspect of the present disclosure, the present disclosure provides use of a formulation in treating cancer. The formulation is used to inhibit or block the aforementioned targets or for inhibiting the expression or activity of OXCT1 protein.

According to an embodiment of the present disclosure, the cancer is at least one selected from liver cancer, pancreatic cancer, colorectal cancer, and breast cancer.

In yet another aspect of the present disclosure, the present disclosure provides a method for treating cancer. According to an embodiment of the present disclosure, the method includes: inhibiting or blocking the aforementioned target in cancer cells or inhibiting an expression or activity of the OXCT1 protein.

According to an embodiment of the present disclosure, the cancer is at least one selected from liver cancer, pancreatic cancer, colorectal cancer, and breast cancer.

### Advantageous Effects of the Present Disclosure

(1) In comparison with the prior art, the present disclosure has found that the succinylation modification level of total protein in cancer tissues of clinical patients with colorectal cancer, breast cancer, or liver cancer is significantly up-regulated compared to corresponding paracancerous tissues. A positive correlation between succinylation modification of total protein and OXCT1 protein expression level was found in cancer cell lines by modulating the expression of protein OXCT1. Further, it is confirmed that the OXCT1 protein can promote the occurrence and progression of liver cancer, colorectal cancer, or breast cancer through its succinyltransferase activity in mouse models, which provides a new theoretical basis and molecular mechanism for future treatment of liver cancer, colorectal cancer or breast cancer.

(2) The Applicant has found that the conventional ketone catabolic enzyme OXCT1 protein has succinyltransferase activity, and the OXCT1 protein can promote the occurrence and progression of colorectal cancer, breast cancer or liver cancer based on the succinyltransferase activity, the succinyltransferase active site thereof is G424, and it exerts the succinyltransferase activity independent of the ketone decomposing function. After inactivation of the ketolytic activity (E344G mutation), the OXCT1 protein has a stronger succinyltransferase activity. It is suggested that it may be an effective target for tumors by targeting the succinyltransferase activity of the OXCT1 protein. Compared with the prior art, the present disclosure provides new targets and products for cancer diagnosis, treatment, and prognosis evaluation, and related products are substances inhibiting the succinyltransferase activity of the OXCT1 protein.

Additional aspects and advantages of the present disclosure will be set forth in part in the description which follows and, in part, will be obvious from the description, or may be learned by practice of the present disclosure.

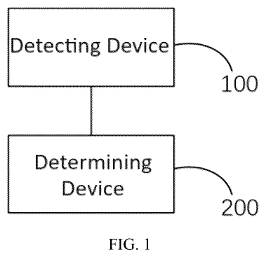

The present disclosure provides a system for determining a source of a sample to be tested. As illustrated in FIG. 1, the system includes a detecting device 100 and a determining device 200. After the sample to be tested enters the detecting device 100, the detecting device 100 can detect a succinyltransferase activity of OXCT1 protein in the sample to be tested. The determining device 200 can then determine the source of the sample to be tested based on the succinyltransferase activity of the OXCT1 protein obtained in the detecting device 100.

Hereinafter, examples of the present disclosure will be described in detail. The examples described below are illustrative only and are not to be construed as limitations of the present disclosure. The unspecified techniques or conditions in the examples are those as described in the literature in the related art or according to the product specifications. The used reagents or instruments without indicating the manufacturer are the conventional and commercially available products.

### Example 1: OXCT1 Upregulating Succinylation Modification Level of Total Protein in Hepatoma Cells

The succinylation modification level of total protein in hepatoma cells with an overexpression of OXCT1 or with OXCT1 knockdown was detected using Western blot.

OXCT1 in hepatoma cell lines HepG2 and Huh7 was overexpressed and knocked down, respectively, and the cells after stable overexpression or knocking down were harvested. The respective components of the cells were broken by the cell lysis solution to allow the cell contents (including proteins) to be incorporated into the cell lysis solution. A quantitative adjustment of the protein was performed on the cell lysis solution under the same conditions, and finally the respective components of the sample with uniform protein content were subjected to Western bolt detection and analysis. Specifically, after separation by SDS-PAGE electrophoresis, the protein was transferred to a nitrocellulose membrane and blocked by skim milk; after the monoclonal antibody (primary antibody) and the pan-succinylated modified monoclonal antibody (primary antibody) that specifically bind to proteins OXCT1 and ACTIN bound thereto sufficiently and then bound to the secondary antibody specifically binding to the primary antibody, chemical development and fixing were performed; and images were scanned and analyzed.

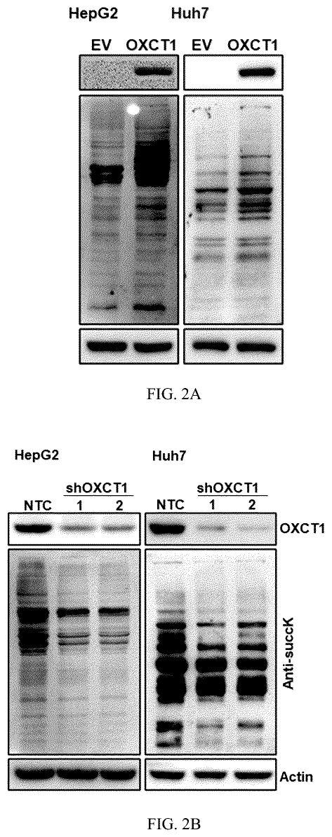

Results are as illustrated in FIG. 2. In the hepatoma cell lines overexpressing OXCT1 (as illustrated in FIG. 2A), the succinylation level of total protein were significantly up-regulated, while the succinylation modification level of total protein was significantly reduced in the hepatoma cell line with OXCT1 knockdown (as illustrated in FIG. 2B). The above cell experiments indicate that the expression level of OXCT1 was positively correlated with the succinylation modification level of protein in hepatoma cells.

### Example 2: Constructing Cell Lines with Stable High Expression of Different OXCT1 Variants

### (1) Constructing Overexpression Plasmids for Different OXCT1 Variants

The cDNA sequence of the CDs region of OXCT1 was amplified with PCR, and the purified CDs sequence of OXCT1 was constructed into a pSIN-lenti-puro expression vector (purchased from Addgene) to obtain OXCT1-overexpressing plasmid pSIN-lenti-puro-OXCT1-WT. On this basis, the DNA sequences of OXCT1 E344G, G424E, and other point mutants were amplified by PCR fragment and were constructed into pSIN-lenti-puro expression vector by homologous recombination method, and the OXCT1 overexpressing mutant plasmid was obtained.

### (2) Transfecting Human Liver Cancer Cells, Breast Cancer Cells, and Colorectal Cancer Cells

The obtained pSIN-lenti-puro-OXCT1-WT, E344G, G424E and the pSIN-lenti-puro empty vectors (as a control) were each transferred to HEK293T cells using PEI (Polysciences). DMEM medium (DMEM; Gibco BRL) was changed after 6 h, and supplemented with 10% fetal bovine serum (Gibco BRL). Lentivirus supernatant was collected 48 h later and infected with human hepatoma cells HepG2 or Huh7 cells, human breast cancer cell line MDA-MB-231, and human colorectal cancer cell line HCT116. The stable cell strain HepG2/Huh7/231/HCT116-OXCT1 with overexpression of OXCT1 and its variants can be obtained by screening for about 10 days with puromycin (0.5 μg/mL; Sigma-aldrich), and the control cell line was HepG2/Huh7/231/HCT116-EV control.

### (3) Culturing Cell Strains Stably and Highly Expressing Different OXCT1 Variants

Hepatoma cells HepG2 or Huh7, human breast cancer cell line MDA-MB-231, and human colorectal cancer cell line HCT116 were cultured with DMEM medium (DMEM; Gibco BRL) supplemented with 10% fetal bovine serum (Gibco BRL) and medium with half inhibitory concentration of puromycin, and cultured in a sterile cell incubator containing 5% carbon dioxide at 37° C.

### Example 3: Mechanism Research of OXCT1 Promoting Succinylation of Proteins

### 1. Identification of OXCT1 Succinylation-Modified Substrates

### (1) 4D Succinylation Protein Profile Identification of OXCT1-Dependent Succinylation-Modified Proteins and Sites

HepG2 cells stably overexpressing OXCT1-WT and control cells (HepG2-EV control) were set up with 3 biological replicates per group and identified by mass spectrometry using 4D succinylated proteomics. The specific procedure was as below: harvesting each group of cells, and breaking each component of cells by cell lysis solution, to incorporate the cell content (including protein) into the cell lysis solution; performing protein quantitative adjustment of cell lysis solution under the same condition; then digesting the protein by trypsin; performing enrichment with pan-succinylated antibody; and finally performing mass spectrometry analysis with Tims-TOF Pro instrument. It was found that OXCT1 promoted succinylation modification of multiple proteins and sites, most notably for LACTB.

### (2) Verification of Mass Spectrometry Results by Cell-Level Co-Immunoprecipitation

10 μg of pSIN-3×Flag-LACTB-WT plasmid and 10 μg of pSIN-HA-OXCT1-WT plasmid were transiently transfected in HEK2293T cells using PEI (Polysciences), DMEM medium (DMEM; Gibco BRL) was changed after 6 h, and supplemented with 10% fetal bovine serum (Gibco BRL). After 48 h, the cells were harvested in accordance with the normal harvesting procedure, resuspended in pre-chilled IP buffer containing 1% NP40, and the cells were rotated and lysed on a spinner for 2 h. Samples were centrifuged at a low temperature, 13,000 rpm, for 15 min, the supernatant was taken for quantification to confirm the protein concentration, and some samples were taken as input for collection and preservation according to the protein collection procedure. The remaining samples were transferred to a new EP tube for pre-clear with the same total protein amount. A suitable volume of A/G beads was added and incubated for 3 h at low temperature on a spinner to reduce the effect of non-specific bands. The time of Pre-clear can be adjusted according to experimental needs. A/G beads need to be rinsed with IP buffer in advance, without blowing, so as not to break the beads. Cryogenic centrifugation was performed at 3,000 rpm for 2 min. The supernatant was carefully transferred to a new EP tube without pipetting the beads. The samples were divided into two equal parts, one for the IgG group and one for the target protein group. IgG antibody was added to the IgG group, the corresponding primary antibody was added to the target protein group, and incubated overnight at low temperature. IgG group antibodies were usually added in an amount of ½ or ⅓ of the target protein. The next day, 20 μl of beads was added and incubated at a low temperature for 2 h. Cryogenic centrifugation was performed at 3,000 rpm for 2 min. The supernatant was pipetted and discarded, and washed under rotation 3 times with 0.5% NP40 in an IP buffer, for 10 min each time. 40 μl of IP buffer was added to mix well with 10 μl 5× loading buffer, the mixture was heated for 5 min at 100° C. in a metal bath and cooled to room temperature.

The sample was analyzed through Western bolt detection, and the specific steps were as below: after separation by SDS-PAGE electrophoresis, the protein was transferred to a nitrocellulose membrane, and blocked by skim milk; after the monoclonal antibodies (primary antibodies) and the pan-succinylated modified monoclonal antibodies (primary antibodies) specifically binding to the four proteins (OXCT1 and ACTIN, LACTB, Flag-Tag) respectively bound thereto sufficiently, and then bound to the secondary antibody specifically binding to the primary antibody, chemical development and fixing were performed; and images were scanned and analyzed.

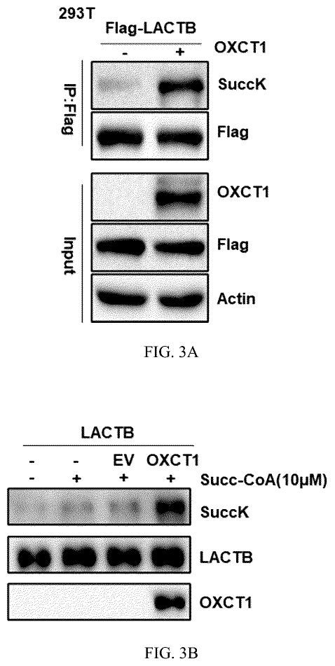

As illustrated in FIG. 3A, in HEK293T cells overexpressing OXCT1, succinylation levels of LACTB were significantly upregulated. Thus, LACTB was a substrate regulated by succinylation modification of OXCT1, indicating reliable mass spectrometry results.

### 2. Identification of OXCT1 Succinyltransferase Activity

### (1) OXCT1, LACTB Protein Induction

The purified CDs sequence of OXCT1 was constructed into pGEX-4T1-GST prokaryotic expression vector (purchased from Addgene) and the purified CDs sequence of LACTB was constructed into pet22 (b) His prokaryotic expression vector (purchased from Addgene). Recombinant plasmids were transferred to Rosetta competent cells, plated, and incubated overnight in an inverted incubator at 37° C. A single colony was picked into a 15 ml centrifuge tube containing LB medium, added with the corresponding volume of antibiotics, and grown overnight at 37° C. The bacterial solution was added into an Erlenmeyer flask containing LB medium at a ratio of 1:50, and incubated at 37° C. on a shaker. After incubation for 2 h, an ultraviolet spectrophotometer was used to detect the absorbance of bacterial solution at the wavelength of 600 nm. When the OD value reached 0.6 to 0.8, the bacterial solution was placed in a chromatography refrigerator at 4° C. for cooling. After 20 min, IPTG with a final concentration of 1 mM was added to the cooled bacterial solution and induced overnight at 16° C. The bacteria liquid was collected the next day and stored at −80° C.

### (2) GST Protein Purification

The frozen bacteria liquid was crushed using an ultrasonic crusher, and the whole process was carried out at a low temperature; the crushed bacteria liquid was quickly frozen in liquid nitrogen, and the ultrasonic was repeated 2-3 times until the bacteria liquid was clear. Cryogenic centrifugation was performed at 12,000 rpm for 15 min. 20 μl of GST beads (purchased from cytiva, 17075601) was added to the bacterial supernatant and incubated for 30 min on a low-temperature spinner. Cryogenic centrifugation was performed at 3,000 rpm for 2 min. The supernatant was pipetted and discarded, rinsed with pre-cooled PBST buffer at low temperature 3 times, for 5 min each time. Cryogenic centrifugation was performed at 3,000 rpm for 2 min. The supernatant was completely pipetted and the GST beads containing the protein of interest were obtained.

### (3) His Protein Purification

The formulation of imidazole buffer was shown in Table 1:

TABLE 1
Imidazole buffer, pH 7.5 (1 L)

| NaCl | 29.22 g |
|---|---|
| Tris | 1.21 g |
| Glycerin | 3% |

The bacterial precipitate from 50 mL of bacterial solution was collected and resuspended with 20 ml of 50 mM imidazole buffer, and the corresponding volume of PMSF and DTT was added. Sonication was performed with an ultrasonic cell disruptor, 6 mm probe, power was 18%, ultrasonic treatment for 1 s, stop 2 s, time was 25 min. The whole process was performed at a low temperature. Cryogenic centrifugation was performed at 10,000 rpm for 15 min. The centrifuged supernatant was transferred to a pretreated nickel column and incubated for 1 h at low temperature with rotation. Cryogenic centrifugation was performed at 3,000 rpm for 1 min. Elution was performed with 200 mM imidazole buffer, at a volume of 5 ml, rotated for 10 min at low temperature for 3,000 rpm for 1 min. The purified protein eluate was concentrated and desalted by passing through a concentration tube.

### (4) Succinyltransferase Assay of Protein In Vitro

The formulation of succinyltransferase reaction buffer was shown in Table 2:

TABLE 2
Reaction Buffer (pH 7.5)

| | | |
|---|---|---|
| HEPES | 10 | mM |
| Na₃VO₃ | 2 | mM |
| Sodium pyrophosphate | 5 | mM |
| NaCl | 300 | mM |

| | |
|---|---|
| Glycerin | 2% |

4 μg of GST-OXCT1 and 5 μg of His-LACTB, which were in vitro purified, were taken, while His-LACTB was used as substrate protein. The reaction system was prepared.

The succinyltransferase reaction system was shown in Table 3:

TABLE 3
Succinyltransferase reaction system (100 μl)

| | | |
|---|---|---|
| GST-tagged protein | 4 | μg |
| His-tagged protein | 4 | μg |
| Succinyl-CoA | 10 | μM |
| Reaction buffer QS to | 100 | μl |

A water bath reaction was performed for 10 min at 37° C., the reaction was stopped quickly by placing it on an ice box, and the mixture was directly added to a 25 μl of 5× loading buffer metal bath, heated for 5 min at 100° C., taking sample, and the change of succinylation level was detected by Western blotting. The specific steps were as below: after separation by SDS-PAGE electrophoresis, the protein was transferred to a nitrocellulose membrane, and the protein was blocked by skim milk; after the monoclonal antibody (primary antibody) and the pan-succinylated modified monoclonal antibody (primary antibody) specifically binding to the two proteins (OXCT1 and LACTB) respectively bound thereto sufficiently, and then bound to the secondary antibody specifically binding to the primary antibody, chemical development and fixing were performed; and images were scanned and analyzed. As illustrated in FIG. 3B, OXCT1 can significantly promote the succinylation modification level of LACTB in the in vitro system, indicating that OXCT1 was able to exert the function of succinyltransferase in vitro to promote the succinylation modification of substrate protein LATCB.

### 3. Identification of OXCT1 Succinyltransferase Active Site

### (1) Prediction of the OXCT1 Succinyltransferase Active Site Using Software and Screening of the Enzyme Active Site in Hepatoma Cell Lines

The docking of OXCT1 with succinyl-CoA was simulated by software, and the amino acid residues of OXCT1 binding with succinyl-CoA were predicted. The results indicate that multiple amino acid residues of OXCT1 had a variety of intermolecular interactions with succinyl-CoA. The amino acid residues interacting with the succinyl group were F64, N90, N91, G138, G324, E344, and G424, which interacted with succinyl-CoA by means of different intermolecular forces. Then, mutations were performed based on the intermolecular force of the interaction with the succinyl group, allowing it to lose the initial intermolecular interaction with the succinyl group. R281 and E369 mutations were used as negative controls. The effects of different OXCT1 variants on the succinylation modification level of protein were detected by Western bolt. After separation by SDS-PAGE electrophoresis, the protein was transferred to nitrocellulose membrane, and the protein was blocked by skim milk; after the monoclonal antibody (primary antibody) and the pan-succinylated modified monoclonal antibody (primary antibody) specifically binding to the two proteins (OXCT1 and ACTIN) respectively bound thereto sufficiently, and then incubated with the secondary antibody specifically binding to the primary antibody, chemical development and fixing were performed; and images were scanned and analyzed.

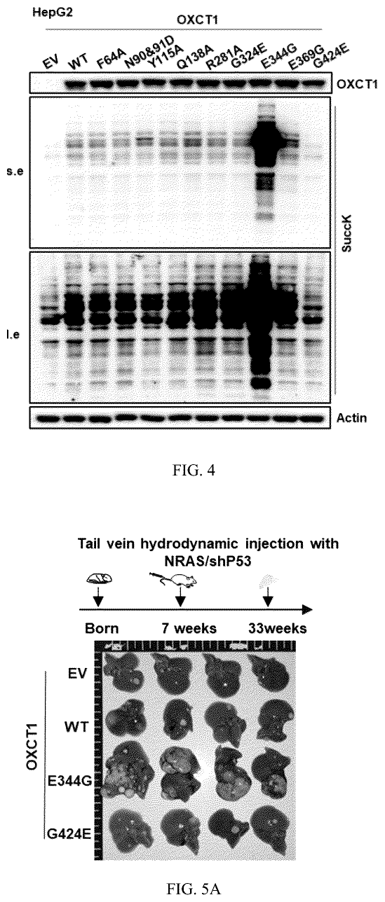

Results as illustrated in FIG. 4, only through the G424 mutation, the succinylation modification level of protein was not up-regulated, while the key site of ketone oxidative decomposition (E344) was mutated and inactivated, the protein succinylation level was increased more significantly. Therefore, the Western blot results showed that the succinylation modification function of OXCT1 did not depend on the oxidative decomposition activity of Ketones, and the function of ketone decomposition of OXCT1 restricted its succinyltransferase activity. After the inactivation of the ketone decomposition enzyme (E344G), the succinyltransferase activity was enhanced. G424 is the key site for OXCT1 to exert succinyltransferase function.

### Example 4: Contribution of Succinyltransferase Activity of OXCT1 to Liver Cancer

### Assay of Induction of Hepatoma In Situ in Liver-Specific Knockout OXCT1 Mice.

Hepatoma in situ of C57BL/6 mice with specific knockout of OXCT1 was induced by hyperbaric tail vein injection of NRAS/shP53/SB13 plasmid (this system can induce hepatoma in situ in mice), and OXCT1 and its variants plasmids (pT-Caggs-EV, pT-Caggs-OXCT1-WT, pT-Caggs-OXCT1-E344G, pT-Caggs-OXCT1-G424E). Ten weeks later, the mice were dissected, the livers of the mice were weighed, and the tumor formation sites and diameters were counted and measured. Liver tumor and paracancerous tissues samples were obtained. The respective tissues were crushed into tissue homogenate by means of a tissue crusher, and then the respective components of the cells were broken by the cell lysis solution to allow the cell contents (including proteins) to be incorporated into the cell lysis solution, the protein was quantified in the cell lysis solution under the same conditions, and finally the respective components of the sample with an equal amount was subjected to Western bolt detection and analysis. The specific steps were as follows: after separation by SDS-PAGE electrophoresis, the protein was transferred to nitrocellulose membrane, and the protein was blocked by skim milk; after the monoclonal antibody (primary antibody) and the pan-succinylated modified monoclonal antibody (primary antibody) specifically binding to the two proteins (OXCT1 and Calnexin) respectively bound thereto sufficiently, and then bound to the secondary antibody specifically binding to the primary antibody, chemical development and fixing were performed; and images were scanned and analyzed.

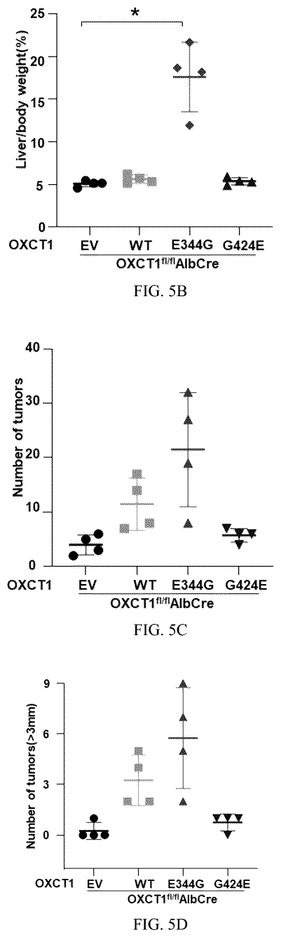

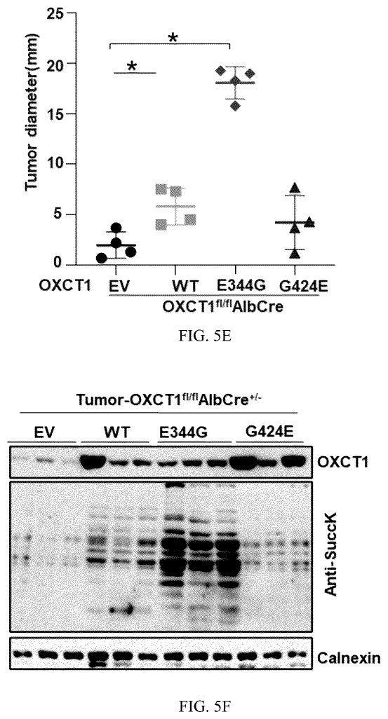

Results are as illustrated in FIG. 5A to FIG. 5F, supplementation of OXCT1 WT and E344G variant significantly promoted the occurrence and progression of liver cancer, and E344G variant had a stronger role in promoting the occurrence and progression of liver cancer, while supplementation of OXCT1 G424E variant could not promote the occurrence and progression of liver cancer. These results suggested that OXCT1, as succinyltransferase, had contribution to the development of liver cancer by regulating the protein succinylation modification.

### Example 5: Upregulation of Succinylation Modification Level of Total Protein by OXCT1 in Colorectal Cancer and Breast Cancer

The succinylation modification level of total protein in colorectal cancer cells and breast cancer cells with an overexpression of OXCT1 or with OXCT1 knockdown was detected using Western blot.

In colorectal cancer cell line HCT116 and breast cancer cell line MDA-MB-231, OXCT1-WT, OXCT1-E344G, OXCT1-G424E was overexpressed or OXCT1 was knocked down, respectively. Thereafter, the cells were harvested, and the respective components of the cells were broken by the cell lysis solution to allow the cell contents (including proteins) to be incorporated into the cell lysis solution. Then, quantitative adjustment of the proteins was performed in the cell lysis solution under the same conditions, and finally the respective components of the sample with uniform protein content was subjected to Western bolt detection and analysis. The specific steps were as follows: after separation by SDS-PAGE electrophoresis, the protein was transferred to nitrocellulose membrane, and the protein was blocked by skim milk; after the monoclonal antibody (primary antibody) and the pan-succinylated modified monoclonal antibody (primary antibody) specifically binding to the two proteins (OXCT1 and ACTIN) respectively bound thereto sufficiently, and then bound to the secondary antibody specifically binding to the primary antibody, chemical development and fixing were performed; and images were scanned and analyzed.

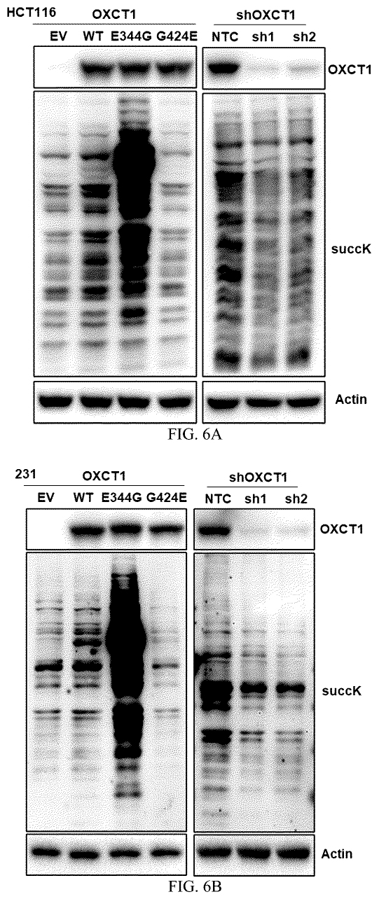

Results are as illustrated in FIG. 6. The succinylation level of total protein was significantly up-regulated in the colorectal cancer cell lines overexpressing the respective OXCT1 variants (as illustrated on the left in FIG. 6A), while the succinylation modification level of total protein was significantly reduced in the colorectal cancer cell line with OXCT1 knockdown (as illustrated on the right in FIG. 6A). The succinylation level of total protein were significantly up-regulated in the breast cancer cell lines overexpressing the respective OXCT1 variants (as illustrated on the left in FIG. 6B), while the succinylation modification level of total protein was significantly reduced in the breast cancer cell line with OXCT1 knockdown (as illustrated on the right in FIG. 6B). All the above cell experiments indicate that the expression level of OXCT1 was positively correlated with the succinylation modification level of protein in the colorectal cancer cells and the breast cancer cells, and OXCT1-E344G could upregulate the succinylation modification level of total protein significantly. In contrast, OXCT1-G424E cannot upregulate the succinylation modification level of total protein.

### Example 6: Contribution of Succinyltransferase Activity of OXCT1 to Colorectal Cancer and Breast Cancer

Cell proliferation in colorectal cancer and breast cancer cells overexpressing or silencing OXCT1 was detected by cytometry.

The changes in cell growth rate of colorectal cancer cell lines and breast cancer cell lines silencing OXCT1 and exogenously overexpressing OXCT1 variants were detected by counting the cell number of different components of cells through the method of blood cell technology. The cells in the experimental groups (HCT116/MDA-MB-231-shOXCT1 and HCT116/MDA-MB-231-OXCT1-WT\E344G\G424E) or the cells in control groups (HCT116/MDA-MB-231-NTC Control and HCT116/MDA-MB-231-EV Control) were inoculated in a 12-well culture plate with a cell density of 1.5×10⁴ cells per well. The cells in the corresponding holes were digested and dispersed into a single cell every 48 h. After diluted to a certain multiple, the cell dilution suspension droplets were added to the blood cell counting plate. The number of cells was counted under the microscope. The number of cells per hole was obtained by multiplying the dilution multiple. The experiment was triplet repeated.

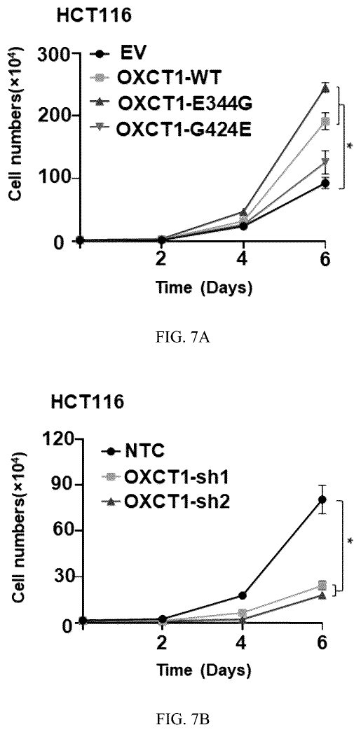

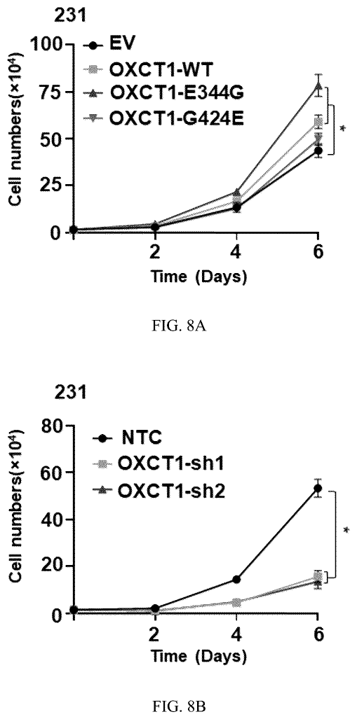

Results are as illustrated in FIG. 7A, FIG. 7B, FIG. 8A, and FIG. 8B. The proliferation of colorectal and breast cancer cells silencing OXCT1 (OXCT1-sh1/2 curves in FIG. 7B and FIG. 8B) was significantly slower than that of control cells, i.e., HCT116/MDA-MB-231-NTC Control and HCT116/MDA-MB-231-EV Control, (NTC and EV curves in FIG. 7A, FIG. 7B, FIG. 8A, and FIG. 8B). In contrast, the proliferation of colorectal and breast cancer cells overexpressing OXCT1 was significantly faster (OXCT1-WT curve in FIG. 7A and FIG. 8A). The proliferation of colorectal and breast cancer cells overexpressing OXCT1-E344G (OXCT1-E344G curves in FIG. 7A and FIG. 8A) was significantly faster. The proliferation of colorectal and breast cancer cell lines overexpressing OXCT1-G424E was not significantly different from that of the controls (OXCT1-G424E curves in FIG. 7A and FIG. 8A). The results of cytometry experiment indicate that, the growth of colorectal cancer cells HCT116 and breast cancer cells MDA-MB-231 was inhibited by silencing OXCT1, while the growth of colorectal cancer cells HCT116 and breast cancer cells MDA-MB-231 was promoted by overexpressing OXCT1-WT. The overexpression of OXCT1-E344G could significantly promote the growth of colorectal cancer cell HCT116 and breast cancer cell MDA-MB-231.

In the specification, references to descriptions of the terms “an embodiment”, “some embodiments”, “examples”, “specific examples”, or “some examples”, etc. mean that a particular feature, structure, material, or characteristic described in connection with the embodiment or example is included in at least one embodiment or example of the present disclosure. In this specification, schematic representations of the above terms are not necessarily directed to the same embodiment or example. Furthermore, the particular features, structures, materials, or characteristics described may be combined in any suitable manner in any one or more embodiments or examples. Furthermore, combinations and combinations of the various embodiments or examples and features of the various embodiments or examples described in this specification can be made by those skilled in the art without departing from the scope of the present disclosure.

While embodiments of the present disclosure are illustrated and described above, it can be understood that the above-described embodiments are illustrative and not restrictive and that those skilled in the art can make changes, modifications, substitutions, and alterations without departing from the scope of the present disclosure.

What is claimed is:

1. A target for treating a cancer, wherein the target is an amino acid G424 of OXCT1 protein.

2. The target according to claim 1, wherein the amino acid G424 is a succinyltransferase active site of the OXCT1 protein.

3. The target according to claim 1, wherein the cancer is selected from cancer species with high expression of the OXCT1 protein.

4. The target according to claim 1, wherein the cancer is at least one selected from liver cancer, colorectal cancer, and breast cancer.

5. A cancer therapeutic formulation, comprising a reagent inhibiting or blocking the target according to claim 1.

6. The formulation according to claim 5, wherein the reagent comprises at least one selected from a siRNA, a neutralizing antibody, and a compound inhibiting expression of OXCT1 protein.

7. The formulation according to claim 5, wherein the cancer is selected from cancer species with high expression of the OXCT1 protein.

8. The formulation according to claim 5, wherein the cancer is at least one selected from liver cancer, pancreatic cancer, colorectal cancer, and breast cancer, and preferably, the cancer is at least one selected from liver cancer, colorectal cancer, and breast cancer.

9. A method for determining a source of a sample to be tested, comprising:
determining the source of the sample to be tested based on a succinyltransferase activity of OXCT1 protein in the sample to be tested.

10. The method according to claim 9, wherein the succinyltransferase activity of the OXCT1 protein in the sample to be tested is determined based on a protein succinylation level in the sample to be tested.

11. The method according to claim 10, wherein the protein succinylation level in the sample to be tested is at least two times a predetermined protein succinylation level and is an indication that the sample to be tested is derived from a cancer tissue.

12. The method according to claim 10, wherein the protein succinylation level in the sample to be tested is detected by at least one of Western blot, immunohistochemistry, immunofluorescence, and enzyme-linked immunosorbent assay.

13. The method according to claim 11, wherein the cancer tissue is at least one selected from a liver cancer tissue, a pancreatic cancer tissue, a colorectal cancer tissue, and a breast cancer tissue; and preferably, the cancer tissue is liver cancer, colorectal cancer, or breast cancer.

14. A system for determining a source of a sample to be tested, comprising:
a detecting device configured to detect a succinyltransferase activity of OXCT1 protein in the sample to be tested; and
a determining device connected to the detecting device, the determining device being configured to determine the source of the sample to be tested based on the succinyltransferase activity of the OXCT1 protein obtained in the detecting device.

15. The system according to claim 14, wherein the detecting device is configured to determine, through obtaining a protein succinylation level in the sample to be tested, the succinyltransferase activity of the OXCT1 protein.

16. The system according to claim 14, wherein the detecting device is configured to perform at least one of Western blot, immunohistochemistry, immunofluorescence, and enzyme-linked immunosorbent assay.

17. The system according to claim 15, wherein the protein succinylation level in the sample to be tested is at least two times a predetermined protein succinylation level and is an indication that the sample to be tested is derived from a cancer tissue.

18. A method for treating cancer, comprising:
inhibiting or blocking the target according to claim 1; or
inhibiting an expression or activity of OXCT1 protein in cancer cells.

19. The method according to claim 18, wherein the cancer is at least one selected from liver cancer, pancreatic cancer, colorectal cancer, and breast cancer.
